(19) United States
(12) Patent Application Publication
(10) Pub. No.: US 2022/0087940 A1
(43) Pub. Date: Mar. 24, 2022

(54) DOSAGE FORM COMPRISING A POLYMERIC MATRIX

(71) Applicant: Evonik Operations GmbH, Essen (DE)

(72) Inventors: Christian MOERS, Langen (DE); Thomas Endres, iphofen (DE); Christian Meier, Darmstadt (DE); Jessica del Rosario Ferrand, Eppertshausen (DE); Jan Hendrik Schattka, Darmstadt (DE); Herbert Jung, Karlstein (DE); Thomas Eurich, Hanau (DE)

(73) Assignee: Evonik Operations GmbH, Essen (DE)

(21) Appl. No.: 17/437,678

(22) PCT Filed: Mar. 5, 2020
(86) PCT No.: PCT/EP2020/055810

§ 371 Date: Sep. 9, 2021

Related U.S. Application Data
(60) Provisional application No. 62/816,605, filed on Mar. 11, 2019.

(51) Int. Cl. A61K 9/20; A61K 31/522

(52) U.S. Cl. CPC A61K 9/204; A61K 9/2095; A61K 31/522

(57) ABSTRACT

A dosage form contains a polymeric matrix, containing one or more polymer(s) and a biologically active ingredient. The polymeric matrix contains 10% by weight or more of the one or more polymer(s). The one or more polymer(s) are polymerized from a monomer mixture containing the monomers (a) 70 to 95% by weight of 2-ethylhexyl methacrylate (EHMA) and ethyl methacrylate (EMA), or 2-ethylhexyl methacrylate (EHMA) and methyl methacrylate (MMA); (b) 0 to 25% by weight of a C₂ to C₆ hydroxy-alkylester of acrylic acid or methacrylic acid; and (c) 2.5 to 20% by weight of a C₂ to C₈ alkyl ester of acrylic acid or of methacrylic acid with a quaternary cationic group in the alkyl group.

### FIELD OF THE INVENTION

The invention is concerned with a dosage form, comprising a polymeric matrix. The dosage form shows a sustained release profile of a biologically active ingredient and is resistant against the influence of ethanol.

### BACKGROUND

WO2008/049657A2 describes the use of (meth)acrylate copolymers in slow-release pharmaceutical forms for reducing the influence of ethanol on active ingredient release. Matrix tablets employing about 20% by weight EUDRAGIT® RS polymer and theophylline are not resistant against ethanol and show a strong acceleration of the active ingredient release in ethanolic media. Matrix tablets employing about 20% by weight EUDRAGIT® RS polymer and diltiazem show only a smooth acceleration of the active ingredient release in ethanolic media in acidic or buffered media, however, the release rate is more than 60% after two hours in acidic medium and too fast for sustained release applications. Furthermore, the glass transition temperature of the EUDRAGIT® RS polymer is with 65° C. comparatively high for many matrix applications, especially for thermal processing in melt extrusion processes.

### SUMMARY OF THE INVENTION

Consumption of medication in combination with alcohol may carry a large risk for the patient. This is especially the case when consumed with sustained release formulations that are meant to release a concentration of an active pharmaceutical ingredient (API) within the therapeutical window over a prolonged period. To enable this release profile, these formulations contain a large amount of API that may cause significant and in some cases fatal side effects if released at once. Alcohol dose dumping describes the phenomenon of unintended rapid API release in the presence of alcohol. To increase patient safety, rugged formulations are required that do not exhibit dose dumping over a fast spectrum of alcohol concentrations.

Alcohol resistance is also an important feature of tamper resistant formulations that impede willful API extraction for e.g. drug abuse.

Conventional sustained release matrix formulations are usually not alcohol resistant having the inherent risk of alcohol dose dumping. Polymers that are inherently resistant to alcohol dose dumping carry the advantage of easier formulation as no additional additives, which may disadvantageously interact with the API or modify the overall release profile in gastric or other media, are required to achieve alcohol resistance.

EUDRAGIT® RS is suitable for matrix applications and confers in some environment even ethanol resistance. However, the glass transition temperature of the EUDRAGIT® RS polymer is with 65° C. comparatively high for many matrix applications, especially for thermal processing in melt extrusion processes. Therefore, there is a need for polymers for matrix applications which confer ethanol resistance but which can be processed at lower temperatures, especially in melt extrusion processes.

The invention is concerned with a dosage form, comprising a core, comprising a polymeric matrix, comprising one or more polymer(s) and a biologically active ingredient, wherein the polymeric matrix comprises 10% by weight or more of one or more polymer(s) and wherein the one or more polymer(s) are polymerized from a monomer mixture comprising the monomers

(a) 70 to 95% by weight of 2-ethylhexyl methacrylate (EHMA) and ethyl methacrylate (EMA) or 2-ethylhexyl methacrylate (EHMA) and methyl methacrylate (MMA),

(b) 0 to 25% by weight of a C₂ to C₆ hydroxy-alkylester of acrylic acid or methacrylic acid,

(c) 2.5 to 20% by weight of a C₂ to C₈ alkyl ester of acrylic acid or of methacrylic acid with a quaternary cationic group in the alkyl group.

### DETAILED DESCRIPTION OF THE INVENTION

Dosage Form

The dosage form is comprising a core, comprising a polymeric matrix, comprising one or more polymer(s) and a biologically active ingredient, wherein the polymeric matrix comprises 10% by weight or more, preferably 15% by weight or more, most preferably 18% by weight or more of the one or more polymer(s) and wherein the one or more polymer(s) are polymerized from a monomer mixture comprising the monomers

(a) 70 to 95% by weight of 2-ethylhexyl methacrylate (EHMA) and ethyl methacrylate (EMA) or 2-ethylhexyl methacrylate (EHMA) and methyl methacrylate (MMA),

(b) 0 to 25% by weight of a C₂ to C₆ hydroxy-alkylester of acrylic acid or methacrylic acid,

(c) 2.5 to 20% by weight of a C₂ to C₈ alkyl ester of acrylic acid or of methacrylic acid with a quaternary cationic group in the alkyl group.

Each “% by weight” range for each monomer may be combined with each weight-% range for another monomer.

Disclosed is also the use of the polymer for preparing a dosage form with a sustained release profile and resistance against the influence of ethanol and the dosage form itself.

The dosage form may be present in the form of pellets, beads, tablets, sachets or capsules filled with pellets, beads, granulates or powders. The polymeric matrix usually forms the core of pellets, beads and tablets. Pellets and beads may be present within sachets and capsules. Capsules may be also filled with milled pellets, beads or tablets in the form of granulates or powders.

Pellets or granules may be used as cores or in compressed tablets. As a rough estimation, pellets may have a size in the range of 50 to 1500, 250 to 1250 μm (average diameter), while coated tablets may have a size in the range of above 1000 μm up to 25 mm (diameter or length). As a rule one can say the smaller the size of the pellet cores are, the higher the pellet coating weight gain needed. This is due to the comparably higher surface area of pellets compared to tablets.

The term pellet-containing tablet or compressed tablet is well known to a skilled person. Such a tablet may have a size of around 5 to 25 mm for instance. Usually, defined pluralities of small active ingredient containing pellets are compressed therein together with binding excipients to give the well-known tablet form. After oral ingestion and contact with the body fluid the tablet form is disrupted and the pellets are set free. The compressed tablet combines the advantage of the single dose form for ingestion with the advantages of a multiple form, for instance the dosage accuracy. In tablets containing comparably low amounts of excipients, preferably talcum but also other excipients, may be used in contrast to pellets.

The term minitablet is well known to the skilled person. A minitablet is smaller than the traditional tablet and may have a size of around 1 to 4 mm. The minitablet is, like a pellet, a single dosage form to be used in multiple dosages. In comparison to pellets, which may be in the same size, minitablets usually have the advantage of having more regular surfaces which can be coated more accurately and more uniformly. Minitablets may be provided enclosed in capsules, such as gelatine capsules. Such capsules disrupt after oral ingestion and contact with the gastric or intestinal fluids and the minitablets are set free. Another application of minitablets is the individual fine adjustment of the active ingredient dosage. In this case the patient may ingest a defined number of minitablets directly which matches to the severeness of the disease to cure but also to his individual body weight. A minitablet is different from pellet-containing compressed tablet as discussed above.

The term sachet is well known to the skilled person. It refers to a small sealed package which contains the active ingredient often in pellet-containing liquid form or also in dry pellet or powder form. The sachet itself is only the package form and is not intended to be ingested. The content of the sachet may be dissolved in water or as an advantageous feature may be soaked or ingested directly without further liquid. The latter is an advantageous feature for the patient when the dosage form shall be ingested in a situation where no water is available. The sachet is an alternative dosage form to tablets, minitablets or capsules.

The term capsule is well known to the skilled person. A capsule is like the sachet a container for pellet-containing liquids or also dry pellets or powders. However, in contrast to the sachet the capsule consists of pharmaceutically acceptable excipients such as gelatine or hydroxypropylmethylcellulose (HPMC) and is intended to be ingested like a tablet. The capsules disrupt after oral ingestion and contact with the gastric or intestinal fluids and the contained multiple units are set free. Capsules for pharmaceutical purposes are commercially available in different standardized sizes.

Polymeric Matrix

The dosage form is comprising a polymeric matrix, comprising one or more polymer(s) and a biologically active ingredient. The polymeric matrix may optionally comprise pharmaceutical or nutraceutical acceptable excipients. The one or more polymer(s) and a biologically active ingredient may add up with the weight of the optionally pharmaceutically or nutraceutically acceptable excipients to 100%.

The polymeric matrix usually forms the core, respectively the inner core of the dosage form. The (inner) core of the dosage form comprises, comprises essentially or consists of the polymeric matrix. The polymeric matrix may be formed by mixing and processing the one or more polymer(s) and a biologically active ingredient and optionally pharmaceutically or nutraceutically acceptable excipients to become the core of the dosage form or as cores which are part of a dosage form, comprising a multitude of such cores.

The polymeric matrix comprises 10% by weight or more, preferably 15% by weight or more, most preferably 18% by weight or more of the one or more polymer(s).

The polymeric matrix may comprise 10 to 99% by weight, preferably 15 to 90% by weight or more, most preferably 18 to 60% by weight or more of the one or more polymer(s).

The one or more polymer(s) and the biologically active ingredient may add up to 11% by weight or more of the weight of polymeric matrix. The one or more polymer(s) and the biologically active ingredient may comprise 11 to 100% by weight of the polymeric matrix.

Biologically Active Ingredients

The dosage form is comprising a core, comprising a polymeric matrix, comprising one or more polymer(s) and a biologically active ingredient. The biologically active ingredient may be preferably an active pharmaceutical ingredient and/or an active nutraceutical ingredient.

The polymeric matrix may comprise 90% by weight or less, preferably 85% by weight or less, most preferably 82% by weight or less of a biologically active ingredient.

The polymeric matrix may comprise 1 to 90% by weight, preferably 10 to 85% by weight, most preferably 40 to 82% by weight of a biologically active ingredient.

Pharmaceutically or Nutraceutically Acceptable Excipients

The polymeric matrix may optionally comprise pharmaceutically or nutraceutically acceptable excipients. Such pharmaceutically or nutraceutically acceptable excipients may be selected from the group of antioxidants, brighteners, binding agents, flavoring agents, flow aids, glidants, penetration-promoting agents, pigments, plasticizers, further polymers, pore-forming agents and stabilizers or any combinations thereof. The polymeric matrix may optionally comprise pharmaceutical or nutraceutical acceptable excipients, wherein the one or more polymer(s) and the biologically active ingredient and the pharmaceutically or nutraceutically acceptable excipients may add up to 100%.

The one or more polymer(s) and the biologically active ingredient may add up to 11% by weight or more of the weight of polymeric matrix. The one or more polymer(s) and the biologically active ingredient may comprise 11 to 100% by weight of the polymeric matrix. The polymeric matrix may optionally comprise 0 to 89% by weight of pharmaceutical or nutraceutical acceptable excipients. The polymeric matrix may optionally comprise 0.1 to 80% of pharmaceutical or nutraceutical acceptable excipients.

The polymeric matrix may comprise, for instance in a matrix tablet, 20 to 70% by weight of pharmaceutical or nutraceutical acceptable excipients, for instance calcium hydrogen phosphate, and 30 to 80% by weight of the one or more polymer(s) and the biologically active ingredient.

The polymeric matrix may comprise, for instance in a matrix tablet, 0.1 to 10% by weight of pharmaceutically or nutraceutically acceptable excipients, for instance Mg stearate, and 90 to 99.9% by weight of the one or more polymer(s) and the biologically active ingredient.

Active Pharmaceutical Ingredients

The biologically active ingredient may be preferably an active pharmaceutical ingredient and/or a active nutraceutical ingredient. The invention is preferably useful for sustained release formulated pharmaceutical dosage forms comprising a pharmaceutical active ingredient.

Therapeutical and chemical classes of active ingredients used in sustained release formulated coated pharmaceutical dosage forms are for instance analgetics, antibiotics or anti-infectives, antibodies, antiepileptics, antigens from plants, antirheumatics, betablocker, benzimidazole derivatives, cardiovascular drugs, chemotherapeutics, CNS drugs, digitalis glycosides, gastrointestinal drugs, e.g. proton pump inhibitors, enzymes, hormones, liquid or solid natural extracts, oligonucleotides, peptidhormones, proteins, therapeutical bacteria, peptides, proteins, urology drugs, vaccines (where applicable, including (metal)salts thereof e.g. aspartates, chlorides, orthates of the mentioned substances).

Further examples of drugs for sustained controlled release may be: acamprosat, aescin, amylase, acetylsalicylic acid, adrenalin, 5-amino salicylic acid, aureomycin, bacitracin, balsalazine, beta carotene, bicalutamid bisacodyl, bromelain, bromelain, budesonide, caffeine citrate, calcitonin, carbamacipine, carboplatin, cephalosporins, cetrorelix, clarithromycin, chloromycetin, cimetidine, cisapride, cladribine, clorazepate, cromalyn, 1-deaminocysteine-8-D-arginine-vasopressin, deramciclane, detirelix, dexlansoprazole, diclofenac, didanosine, digitoxin and other digitalis glycosides, dihydrostreptomycin, dimethicone, divalproex, drospirenone, duloxetine, enzymes, erythromycin, esomeprazole, estrogens, etoposide, famotidine, fluorides, garlic oil, glucagon, granulocyte colony stimulating factor (G-CSF), heparin, hydrocortisone, human growth hormone (hGH), ibuprofen, ilaprazole, insulin, Interferon, Interleukin, Intron A, ketoprofen, lansoprazole, leuprolidacetat lipase, lipoic acid, lithium, kinin, memantine, mesalazine, methenamine, methylphenidate, metoprolol succinate, milameline, minerals, minoprazole, naproxen, natamycin, nitrofurantion, novobiocin, olsalazine, omeprazole, orothates, pancreatin, pantoprazole, parathyroidhormone, paroxetine, penicillin, perprazol, pindolol, polymyxin, potassium, pravastatin, prednisone, preglumetacin progabide, pro-somatostatin, protease, quinapril, rabeprazole, ranitidine, ranolazine, reboxetine, rutosid, somatostatin streptomycin, subtilin, sulfasalazine, sulphanilamide, tamsulosin, tenatoprazole, thrypsine, valproic acid, vasopressin, vitamins, zinc, including their salts, derivatives, polymorphs, isomorphs, or any kinds of mixtures or combinations thereof.

A further example for a pharmaceutical active ingredient may be theophylline (as used in the examples).

Active Nutraceutical Ingredients

Nutraceuticals are well known to the skilled person. Nutraceuticals are often defined as extracts of foods claimed to have medical effects on human health. Thus, nutraceutical active ingredients may display pharmaceutical activities as well: Examples for nutraceutical active ingredients may be resveratrol from grape products as an antioxidant, soluble dietary fiber products, such as psyllium seed husk for reducing hypercholesterolemia, broccoli (sulphane) as a cancer preservative, and soy or clover (isoflavonoids) to improve arterial health. Thus, it is clear that many substances listed as nutraceuticals may also be used as pharmaceutical active ingredients.

Depending on the territory, the specific application, the local authority legislation and classification, the same substance may be listed as a pharmaceutical or as an active neutraceutical ingredient respectively as a pharmaceutical or a nutraceutical composition or even both. Thus, it is evident to a skilled person that there is a broad overlap between the terms pharmaceutical or an active neutraceutical ingredient respectively a pharmaceutical or a nutraceutical composition.

Nutraceuticals or nutraceutical active ingredients are sometimes defined as extracts of foods claimed to have medical effects on human health.

Nutraceuticals or nutraceutical active ingredients may also include probiotics and prebiotics. Probiotics are living microorganisms believed to support human or animal health when consumed, for example certain strains of the genera Lactobacillus or Bifidobacterium. Prebiotics are nutraceuticals or nutraceutical active ingredients that induce or promote the growth or activity of beneficial microorganisms in the human or animal intestine.

The active neutraceutical ingredient may be usually contained in a medical format such as capsule, tablet or powder in a prescribed dose. Examples for nutraceuticals are resveratrol from grape products or anthocyanines from blueberries as antioxidants, soluble dietary fiber products, such as psyllium seed husk for reducing hypercholesterolemia, broccoli (sulphane) as a cancer preservative, and soy or clover (isoflavonoids) to improve arterial health. Other nutraceuticals examples are flavonoids, antioxidants, alpha-linoleic acid from flax seed, beta-carotene from marigold petals or antocyanins from berries. Sometimes the expression neutraceuticals may be used as synonym for nutraceuticals.

Monomers (a)

Preferred monomers (a) are 2-ethylhexyl methacrylate (EHMA) and ethyl methacrylate (EMA) and/or methyl methacrylate (MMA). Most preferred is the combination of 2-ethylhexyl methacrylate (EHMA) with ethyl methacrylate (EMA) or of 2-ethylhexyl methacrylate (EHMA) with methyl methacrylate. 2-ethylhexyl methacrylate (EHMA) and ethyl methacrylate (EMA) or 2-ethylhexyl methacrylate (EHMA) and methyl methacrylate (MMA) may be included as monomers (a1) and (a2). The ratio by weight of 2-ethylhexyl methacrylate (EHMA):ethyl methacrylate (EMA) or of 2-ethylhexyl methacrylate (EHMA):methyl methacrylate (MMA) may be preferably in the range from 5:1 to 1:1, from 4:1 to 1.5:1 or from 3.5:1 to 2:1.

Monomers (b)

C₂ to C₆ or C₂ to C₄ hydroxy-alkylesters of acrylic acid or methacrylic acid (b) are for instance 2-Hydroxyethyl methacrylate, 2-Hydroxypropyl methacrylate, 3-Hydroxypropyl methacrylate, 2,3-Dihydroxypropyl methacrylate, 2-Hydroxyethyl acrylate, 2-Hydroxypropyl acrylate, 3-Hydroxypropyl acrylate, 2,3-Dihydroxypropyl acrylate or any mixture thereof.

The C₂ to C₆ or C₂ to C₄ hydroxy-alkylesters of acrylic acid or methacrylic acid (b) are preferably selected from 2-Hydroxyethyl methacrylate (HEMA).

Monomers (c)

The C₂-C₈ alkyl ester of acrylic acid or of methacrylic acid with a quaternary cationic group, preferably a quaternary ammonium group, in the in the alkyl group (c) may be preferably 2-trimethylammonium ethyl methacrylate chloride (TMAEMC) or 2-trimethylammonium propyl methacrylate chloride (TMAPMC).

### Preferred Embodiments

In a preferred embodiment the dosage form may comprise a polymeric matrix, comprising one or more polymer(s) and a biologically active ingredient, wherein the one or more polymer(s) are polymerized from a monomer mixture comprising the monomers
(a1) 40 to 80, preferably 50 to 75% by weight 2-ethylhexyl methacrylate,
(a2) 10 to 30, preferably 12 to 28% by weight 2-ethyl methacrylate or methyl methylacrylate or both,
(b) 0 to 20, preferably 2.5 to 18% by weight 2-hydroxyethyl methacrylate,
(c) 2.5 to 20, preferably 2.5 to 18, most preferably 2.5 to 15% by weight % 2-trimethylammonium ethyl methacrylate chloride,
wherein (a1), (a2), (b) and (c) add up to 90 to 100, preferably to 100%.

In a preferred embodiment the dosage form may comprise a polymeric matrix, comprising one or more polymer(s) and a biologically active ingredient, wherein the one or more polymer(s) are polymerized from a monomer mixture comprising the monomers
(a1) 50 to 75% by weight 2-ethylhexyl methacrylate,
(a2) 15 to 30% by weight 2-ethyl methacrylate
(b) 0 to 20% by weight 2-hydroxyethyl methacrylate,
(c) 2.5 to 20, preferably 2.5 to 18, most preferably 2.5 to 15% by weight % 2-trimethylammonium-ethyl-methacrylate-chloride,
wherein (a1), (a2), (b) and (c) add up to 90 to 100, preferably to 100%.

In a preferred embodiment the dosage form may comprise a polymeric matrix, comprising one or more polymer(s) and a biologically active ingredient, wherein the one or more polymer(s) are polymerized from a monomer mixture comprising the monomers
(a1) 65 to 75% by weight 2-ethylhexyl methacrylate,
(a2) 10 to 30% by weight methyl methylacrylate
(b) 2.5 to 10% by weight 2-hydroxyethyl methacrylate,
(c) 2.5 to 15% by weight 2-trimethylammonium ethyl methacrylate chloride,
wherein (a1), (a2), (b) and (c) add up to 90 to 100, preferably to 100%.

In a preferred embodiment the dosage form may comprise a polymeric matrix, comprising one or more polymer(s) and a biologically active ingredient, wherein the one or more polymer(s) are polymerized from a monomer mixture comprising the monomers
(a1) 55 to 75% by weight 2-ethylhexyl methacrylate,
(a2) 10 to 30% by weight methyl methylacrylate
(b) 10 to 20% by weight 2-hydroxyethyl methacrylate,
(c) 2.5 to 15% by weight 2-trimethylammonium ethyl methacrylate chloride,
wherein (a1), (a2), (b) and (c) add up to 90 to 100, preferably to 100%.

Minimum Film Forming Temperature (MFFT)

Preferably, the minimum film forming temperature (MFFT) of the one or more polymer(s) is 35° C. or lower, 30° C. or lower, 28° C. or lower, 20° C. or lower or 15° C. or lower.

Preferably, the one or more polymer(s) show a minimum film forming temperature (MFFT) of 3 to 35, 8 to 30, 9 to 26, 15 to 28° C.

The MFFT may be determined according to the Standard of the International Organisation for Standardization DIN ISO 2115 with the exception of point 6.1 in that the maximum difference of the most distant metering points is set to 50° C.

Midpoint glass transition temperature (T_{mg})

Preferably, the midpoint glass transition temperature (T_{mg}) of the one or more polymer(s) may be in the range of 0 to 50, 10 to 40, 15 to 38 or 20 to 36° C.

DSC measurement of the dry polymer substance was conducted according to DIN EN ISO 11357-2 with a heating rate of 20° C./min. The midpoint glass transition temperature T_{mg} was determined by half step height method as described in section 10.1.2 of DIN EN ISO 11357-2.

Molecular Weight Mw—Polydispersity Index

Preferably the weight average molecular weight M_{w} of the one or more polymer(s) as disclosed is from 10,000 to 200,000, 50,000 to 150,000, 60,000 to 140,000, 70,000 to 130,000, 80,000 to 120,000 or 85,000 to 110,000 Dalton.

The polydispersity index may be determined by calculation of the Mw/Mn ratio (weight average molecular weight/number average molecular weight (determined by GPC)). The polydispersity index of the inventive polymer may be in the range from 1.2 to 4.0, 1.3 to 3.0, 1.5 to 2.5 or from 1.6 to 2.3.

Gel permeation chromatography (GPC) may be used to determine the number- and weight-average molecular weights (Mₙ, M_{w}) and the polydispersity (D) of the inventive polymers as disclosed according to DIN 55672-1. Equipment consisted of four PSS SDV columns (Mainz, Germany) plus pre-column of the same type, a column oven operating at 35° C., an Agilent (Series 1100, Santa Clara, USA) pump plus RI-detector of the same series. A 0.02 M solution of 2-(diethylamino)ethylamine (DEAEA) in Tetrahydrofuran (THF) was used as eluent at a flow rate of 1 mL/min. Samples were dissolved in the eluent at concentrations of 2 mg/mL. For each measurement 100 μL polymer solution is injected. The values for Mn and Mw may be calculated based on calibration curves generated by poly(methyl methacrylate) standards.

EUDRAGIT® reference samples were measured using the eluent N,N-dimethylacetamide (DMAc). Method for EUDRAGIT® RL/RS is described in more detail by Adler M. et al. (e-Polymers, ISSN (Online) 1618-7229, ISSN (Print) 2197-4586, DOI: https://doi.org/10.1515/epoly.2005.5.1.602).

Process for Preparing the One or More Polymer(s)

A process for preparing the one or more polymer(s) disclosed herein may comprise the polymerization from the monomer mixture in the presence of a polymerization initiator and optionally a chain transfer agent by bulk polymerization, suspension polymerization or emulsion polymerization.

The one or more polymer(s) are preferably (meth)acrylate copolymers and may be produced by radical polymerisation of the monomers in the presence of polymerisation initiators such as ammonium peroxodisulfate.

A Chain transfer agent may be added to improve the process stability and reproducibility of the molecular weight (Mw). However, the Chain-transfer agent may be omitted in many cases, without affecting the properties according to the invention.

Preparation methods for the polymers are known to the expert in the field. Typically, emulsion polymerization, solution polymerization or bulk polymerization will be applied; the preferred preparation of the polymer is by emulsion polymerization.

If emulsion polymerization is used, the operation may advantageously be carried out by the monomer emulsion feed process or the monomer feed process, respectively. For this, water is heated to the reaction temperature in the polymerization reactor. Surfactants and/or initiators may be added at this stage. Then, depending on the mode of operation, the monomer, a monomer mixture or an emulsion of either are fed to the reactor. This dosed liquid may contain initiators and/or surfactants or the initiator and/or the surfactant may be dosed in parallel.

Alternatively, all monomers can be charged into the reactor before adding the initiator. This method is often referred to as batch process.

It is also possible to do a combination of both processes, by polymerizing a part of the monomers in the manner of a batch process, and feeding the other part afterwards.

As known to the expert in the field, the type of process and mode of operation can be chosen, to achieve the desired particle size, sufficient dispersion stability, a stable production process and so on.

Emulsifiers

Emulsifiers, which may be used are especially anionic and non-ionic surfactants. The amount of emulsifier used is generally not more than 5% by weight, preferably in the range of 0.1 to 4% by weight based on weight of the monomer mixture.

Typical emulsifiers are for example alkyl sulfates (e.g. sodium dodecyl sulfate), alkyl ether sulfates, dioctyl sodium sulfosuccinate, polysorbates (e.g. polyoxyethylene (20) sorbitan monooleate), nonylphenol ethoxylates (nonoxynol-9) and others.

Polymerization Initiators

Beside those initiators conventionally used in emulsion polymerization (e.g. per-compounds, such as ammonium peroxodisulfate (APS)) redox systems, such as sodium disulphite-APS-iron may be applied. Also water-soluble azo-initiators may be applied and/or a mixture of initiators can be used. The amount of polymerization initiator may be around 0.005 to 0.5, 0.05 to 0.2, 0.01 to 0.1% by weight, based on total weight of the (meth)acrylate monomers.

Chain-Transfer Agents

Chain-transfer agents are well known to the skilled person and used for controlling the molecular weight and weight distribution in a polymerization process.

A Chain-transfer agent may be added to the monomer mixture before or during the polymerization. Up to 5, up to 4, up to 3, up to 2, up to 1% by weight or 0.05 to 5, 0.1 to 4, 0.2 to 3, 0.25 to 2, 0.1 to 1, 0.05 to 0.5, 0.1 to 0.4% by weight of a Chain transfer agent, calculated on the total weight (100%) of the monomers, may be added to the monomer mixture. It is also possible to add no Chain-transfer agent at all (0%).

A suitable chain-transfer agent may be 2-ethylhexyl thioglycolat (TGEH) or n-butyl mercaptan, n-dodecylmercaptan or 2-mercaptoethanol or any mixtures thereof.

Polymerization Temperature

A suitable polymerization temperature may be in the range of 25 to 120, 30 to 100 or from 50 to 95° C. The polymerization temperature may depend on the initiators within certain limits. For example, if APS is used it is advantageous to operate in the range from 60 to 90° C.; if redox systems are used it is also possible to polymerize at lower temperatures, for example in the range of 25 to 45° C., for instance at 30° C.

Average Particle Size

The average particle size of the polymer particles produced in the emulsion polymerization may range from 10 to 1000, 20 to 500 or 50 to 250 nm. The average particle size of the polymer particles may be determined by methods well known to a skilled person for instance by the method of laser diffraction. The particle size may be determined by laser diffraction, using a Mastersizer 2000 (Malvern). The values can be indicated as particle radius rMS [nm], which is half of the median of the volume based particle size distribution d(v,50).

The obtained dispersion can directly be used to prepare the coating suspension, or—in rare cases—be used as coating suspension without even adding further ingredients.

The dispersion can also be dried, preferably by spray drying, freeze drying or coagulation. Thus a solid can be obtained, which offers certain advantages with regard to handling and logistics.

The dried polymer may then be transferred into a coating suspension by redispersing the solid in water, e.g. (where required) by the use of a high shear mixer.

The dried polymer may also be dissolved in a solvent, e.g. an organic solvent, to prepare a matrix formulation, for instance by spray drying after dissolving a biologically active ingredient.

If coating with coating solutions is preferred, the preparation of the polymer by solution polymerization or bulk polymerization may be a good option, too.

Sustained or Extended Release Pharmaceutical or Nutraceutical Composition

The dosage form as disclosed herein is preferably a pharmaceutical or nutraceutical dosage form, preferably a sustained release or extended release pharmaceutical or nutraceutical dosage form.

The sustained or extended release of the active pharmaceutical or active neutraceutical ingredient may be defined in that the active ingredient release under in-vitro conditions after 2 hours at pH 1.2 in simulated gastric fluid according to USP (for instance USP 32) and subsequent change of the medium to buffered medium of pH 6.8 according to USP may be for instance in the range of 20 to 98, 30 to 90, 40 to 80% in a total time of 4 to 12 or 4 to 8 or 6 to 10 hours, including the 2 hours of the pH 1.2 phase.

Process for Preparing a Dosage Form

Disclosed is a process for preparing a dosage form as described herein by mixing the one or more polymer(s), the biologically active ingredient and optionally pharmaceutically acceptable excipients, processing the mixture by dry granulation, powder compression, spray granulation, wet granulation and extrusion or melt extrusion, comminution to granulates or powders to the final dosage form in the form of pellets, beads, tablets, sachets or capsules filled with such pellets, beads, granulates or powders. Tablets can be obtained from compression of, e.g., powders or granulates.

Use of One or More Polymer(s), Polymerized from a Monomer Mixture Comprising the Monomers

Disclosed is the use of the one or more polymer(s), as disclosed herein, polymerized from a monomer mixture comprising

(a) 70 to 95% by weight of 2-ethylhexyl methacrylate (EHMA) and ethyl methacrylate (EMA) or 2-ethylhexyl methacrylate (EHMA) and methyl methacrylate (MMA),

(b) 0 to 25% by weight of a C₂ to C₆ hydroxy-alkylester of acrylic acid or methacrylic acid,

(c) 2.5 to 20% by weight of a C₂ to C₈ alkyl ester of acrylic acid or of methacrylic acid with a quaternary cationic group in the alkyl group, for preparing a dosage form, comprising a core, comprising a polymeric matrix, as disclosed, with a sustained release profile and resistance against the influence of ethanol.

Ethanol Resistant Composition

The dosage form as disclosed herein is an ethanol (EtOH) resistant composition, preferably an ethanol (EtOH) resistant pharmaceutical or nutraceutical composition.

Ethanol resistant shall mean that the release of a biologically active ingredient, preferably a pharmaceutical or nutraceutical active ingredient, under in-vitro conditions at pH 1.2 for 2 hours in simulated gastric fluid according to USP and subsequent buffer pH 6.8 without the addition of ethanol does not differ by more than plus/minus 20, preferably plus/minus 10% (absolute percentage) in the same media but with the addition of 5, 10, 20 or 40% (w/w) ethanol in the pH 1.2 medium only.

To give an example, if the release rate of the pharmaceutical or active neutraceutical ingredient is in the medium without ethanol is for instance 60%, then the active ingredient release in the same medium with ethanol shall be in the range from 40 to 80% (+/−20% deviation).

Ethanol resistant dosage forms as defined herein are formulations with release kinetics in pH 1.2 medium and subsequent pH 6.8 medium not significantly affected by the presence of ethanol in a pH 1.2 medium. Ethanol resistance may be an important registration requirement in the near future. Conventional pharmaceutical compositions, if coated or uncoated, are usually not resistant to alcohol at all. An ethanol resistant formulation is sometimes also called a rugged formulation.

Resistance against the influence of ethanol (ethanol resistant dosage form) may be defined in that the release profile determined under in-vitro conditions at pH 1.2 and/or at pH 6.8 in a buffered medium according to USP with the addition of 40% (w/w) ethanol is not accelerated by more than 20%, preferably by not more than 10%, and not delayed by more than 20%, preferably by not more than 10%, under the influence of the 40% ethanol containing medium in comparison to a release profile determined in the same medium without ethanol. Generally an acceleration of a release profile is more critical than a delay. Therefore, the upper limit for an acceleration of the release profile is preferably not more than 10%, more preferably not more than 5%, even more preferably there is no acceleration of the release profile at all.

Depending on the certain dosage form the applicable conditions of the USP test may vary for instance if the paddle or basket method has to be used or the stirring has to be 50, 100 or 150 rpm. For the determination of the ethanol resistance it does not matter which USP test is applied for the certain pharmaceutical composition as long as it is the relevant test for the certain pharmaceutical (or nutraceutical) composition and the test conditions with and without ethanol are the same.

Resistance against the influence of ethanol in the sense of the present invention shall be tested in a relevant period of the release of the active ingredient, where meaningful results can be expected. The period which is meaningfully chosen is from or between 10 to 80% of the total dosage release in the medium without ethanol. In this period the resistance against the influence of ethanol shall be determined at a number n of at least n=3, but preferably more than 3, for instance n=4, 5, 6, 7, 8, 9, 10, 11 or 12 uniformly distributed test points. The number of meaningfully chosen test points depends on the total time period of the release profile from or between 10 to 80% of the total dosage release. The longer the time period the more uniformly distributed test points can be chosen meaningful. The first test point should be the first full hour or half hour time point at or after the 10% release point. The last test point should be at the last full hour or half hour time point at or before the 80% release point. The other test point or test points should be in the middle (n=3) or uniformly distributed (n>3) at full hour or half hour time points at or in between the 10 and 80% release phase. The percentage of acceleration or delay is calculated by the arithmetic mean (arithmetic average) of the n values to give the arithmetic mean release.

The term “and/or” in “under in-vitro conditions at pH 1.2 and/or at pH 6.8” means that there may be different meaningful conditions for different pharmaceutical (or nutraceutical) compositions. Resistance against the influence of ethanol shall be determined only in a relevant period of the release of the active ingredient.

Sustained release pharmaceutical compositions have periods of the release of the active ingredient for instance from 6 to 12 or even more hours, with usually more than 10% release within the first two hours at pH 1.2. In this case it is meaningful to test under in-vitro conditions at pH 1.2 and at pH 6.8.

The percentages of acceleration or delay under the influence of the 5, 10, 20 or 40% ethanol containing pH 1.2 medium are calculated by subtraction of corresponding single release values and the calculation of the arithmetic average thereof. The n release values taken from the media (pH 1.2 and subsequent pH 6.8) with ethanol in the pH 1.2 medium are subtracted by the corresponding n release values from the media without ethanol in the pH 1.2 medium and the arithmetic average of the differences is calculated. A positive result stands for an acceleration of the release; a negative result stands for a delayed release.

A dosage form which fulfils these conditions can be considered to be resistant against critically accelerated release or delay of the active compound by thoughtlessness or by addictive behaviour of the patients with respect to the use of ethanol or ethanol-containing drinks. This situation relates essentially to the simultaneous or subsequent consumption of an alcoholic drink together with the taking of the controlled release pharmaceutical form, such that the pharmaceutical form is exposed to a strong ethanol-containing medium in the stomach or intestine.

However, the purpose of the present invention is expressively not to stimulate, to promote or to make possible the consumption of ethanol-containing drinks together with delayed-release pharmaceutical forms, but to alleviate or to avoid the possibly fatal consequences of intentional or inadvertent misuse or abuse.

### Calculation Example 1

If the arithmetic average calculated from the active ingredient release in the medium with ethanol and without ethanol is 8% (=plus 8%), then there is an acceleration caused by the influence of ethanol of 8%. In this case the controlled release pharmaceutical composition is regarded to be resistant against the influence of ethanol because it is within the limit of not more than 20% acceleration.

### Calculation Example 2

If the arithmetic average calculated from the active ingredient release in the medium with ethanol and without ethanol is −23% (=minus 23%), then there is a delay caused by the influence of ethanol of 23%. In this case the controlled release pharmaceutical composition is not regarded to be resistant against the influence of ethanol because it is out of the limit of not more than 20% delay.

### EXAMPLES

### Example 1: Emulsion Polymerization

The procedure is described exemplarily for Polymer 1 (see Table 1). All other polymers were manufactured in the same manner. Setup consisted of a 1 L reaction vessel equipped with lid, agitator, condenser, nitrogen inlet and thermal sensor. Heating was carried out by a thermostat controlled water bath. A dosage pump with silicone tubes was used to dose monomer emulsion into the reaction mixture. In a first step, 534.0 g of water and 6.6 g of sodium dodecyl sulfate (SDS 15, 15.0% (w/w) aqueous solution) were dosed into the reactor, purged with nitrogen and the mixture was then heated to 80° C. In parallel, in a separate flask, monomer emulsion was prepared by mixing 21.3 g of SDS 15, 0.8 g of chain transfer agent (2-Ethylhexylthioglycolat, TGEH), 188.7 g (67.4% (w/w)) EHMA, 63.3 g (22.6% (w/w)) EMA, 14.0 g (5% (w/w)) HEMA, and 14.0 g (5.0% (w/w)) TMAEMC with 76.0 g of water. Stable emulsion was formed by stirring for 20 min. As soon as reaction mixture reached target temperature (80° C.), 6.0 mL of APS initiator (ammonium persulfate, 10% (w/w) aqueous solution) were pipetted into the reactor, followed by feeding of previously prepared monomer-emulsion. Feeding was carried out stepwise using two different rates (10 min at 1.5 g/min, followed by 120 min at 3.0 mg/min). During dosing, reaction temperature was held constant between 80 and 82° C. After complete monomer addition, reaction mixture was stirred for 30 min at 80° C. and then allowed to cool down to room temperature. In total 28.0 g SDS 15 solution were used (4.2 g SDS, 1.5% (w/w) based on polymer weight). Theoretic solid content of the resulting polymer dispersion is 30% (w/w). Dispersion was finally filtered through a 250 μm gauze. Filtrate and polymer coagulate in the reactor were collected and dried for gravimetric analysis. Experimental solid content of the final dispersion was 29.1% (w/w), coagulate was <0.1%.

Table 1 summarizes the compositions of polymers 1-5 (according to the invention), polymer 6 (comparative, not according to the invention) and commercially available polymer EUDRAGIT® RS (comparative, not according to the invention) with sustained release characteristics.

Abbreviations in table 1: (%=% by weight, Da=Dalton, M_{w}=weight-average molecular weight, T_{mg}=midpoint glass transition temperature, MFFT=minimum film forming temperature, D=Dispersity Index)

### Example 2: Preparation of Matrix Tablets

To 240 g of a dispersion of polymer 1 (see example 1) 120 g of deionized water were added and the mixture was stirred for several minutes and passed through a 260 μm sieve. Spray granulation was performed using a Hüttlin Microlab H00263 setup with a 0.8 mm nozzle with bottom spray. 300 g of Theophylline powder were placed in the setup and spray granulation was performed over 1 hour using the following condition until 300 g of the above dispersion were added (corresponds to 60 g of dry polymer):

Inlet air temperature at process start: 35° C.

Inlet air temperature at process end: 44° C.

Inlet air volume 26 m³/h

Inlet air humidity: 73-75% relative humidity (r.h.)

Exhaust air humidity: 73-76% r.h.

Product bed temperature: 22-23° C.

Spray rate of dispersion: 5.9-6.7 g/min

Nozzle pressure: 0.7 bar

Micro climate: 0.4 bar

Filter cleaning interval: every 1 second for 0.2 seconds

Subsequently, the material was passed through a 1.0 mm metal sieve and dried at 40° C. for 24 hours in a drying oven. 0.5 weight-% (1.6 g) of magnesium stearate was added and mixing was performed for 10 minutes using a Turbula® T 10 B 3D shaker mixer (WILLY A. BACHOFEN GMBH, Nidderau-Heldenbergen, Germany). The material was sieved using a 250 μm metal sieve and the fraction <250 μm was used for tableting.

Tablet pressing was performed using an ERWEKA EP-1 lab press (ERWEKA GmbH, Heusenstamm, Germany) applying a pressing force of 3.4 to 4.4 kN. This yielded concave tablets (Punch diameter: 10 mm; curvature radius: 13.6 mm) with a typical thickness of 4.2 mm and crushing strength in the range of 100 to 150 N as measured using an ERWEKA Multicheck (ERWEKA GmbH, Heusenstamm, Germany) and a tablet weight of about 300 mg (+/−4%).

Matrix tablets from polymers 2-6 and EUDRAGIT® were prepared in the same way.

### Example 3: Dissolution Testing in Pure Media and Hydroalcoholic Media

In vitro drug release of the matrix tablets from Example 2 was tested in triplicates using USP I (basket) apparatus. Measurement was carried out at 150 RPM in 900 mL dissolution vessels. Dissolution was tested in 0.1 N HCl (pH 1.2) with and without 40% (w/w) EtOH for 2 h. Subsequently, medium was fully replaced by pH 6.8 EP buffer (without ethanol) and drug release was monitored for another 8 h. API concentration was quantified via UV/VIS spectroscopy. Results are presented in tables 2 and 3 as mean average, relatively to the total drug concentration in the respective vessel after homogenization.

### Example 4: Freeze-Drying of the Dispersion of Polymer 2

The dispersion of Polymer 2 was freeze-dried using a Christ Alpha 1-4 LDplus laboratory freeze drier (Martin Christ Gefriertrocknungsanlagen GmbH, Osterode am Harz, Germany) to obtain a solid material. After freeze-drying, the material was passed through a 250 μm sieve.

### Example 5: Direct Compression

150.05 g of theophylline powder were mixed with 30.02 g of solid Polymer 2 obtained by freeze-drying as described in Example 4 and 0.94 g of magnesium stearate. Mixing was performed in a Turbula orbital shaker T 2 B for 10 min. This mixture was used for tableting on an ERWEKA EP-1 lab press (ERWEKA GmbH, Heusenstamm, Germany).

Tablet pressing was performed using an ERWEKA EP-1 lab press (ERWEKA GmbH, Heusenstamm, Germany) with manual filling applying a pressing force of 3.3 to 4.9 kN. (Punch diameter: 10 mm; curvature radius: 13.6 mm). This yielded concave tablets with a typical thickness of 4.2 mm and crushing strength in the range of 60 to 100 N as measured using an ERWEKA Multicheck (ERWEKA GmbH, Heusenstamm, Germany) and a tablet weight of about 300 mg (+/−4%). The results of the dissolution testing of the polymer 2/direct compressed tablet is shown in table 3.

### Example 6: Rheological Measurements

For rheological measurements, polymer plates with a diameter of 25 mm and a thickness if 1.5 mm were prepared on a Haake Mini Jet via injection molding. For EUDRAGIT® RS PO, the form was heated to 65° C. and the cylinder to 120° C. For polymer 2, the form was heated to 55° C. and the cylinder to 100° C.

Rheological measurements were performed using a MCR 302 (by Anton Paar GmbH, Graz, Austria) under nitrogen atmosphere. A plate-plate geometry with a diameter of 25 μm was used. Heating rate was set to 2 K/min, frequency to 1 Hz and amplitude of deformation to 0.2%. Table 4 lists the respective results, showing that a suitable viscosity window for extrusion opens up for Polymer 2 already at significantly lower temperatures compared to EUDRAGIT® RS PO (PO=powder product). For example, for Polymer 2, viscosities below 10⁷ mPa*s are already reached at above 118° C. while this requires temperatures of 140° C. or more for EUDRAGIT® RS PO (see Table 4). Being able to extrude at lower temperatures is advantageous as, e.g., thermal degradation of APIs will be prevented. Additionally, when extruding at a given temperature, less mechanical energy, i.e. torque, is required to perform the extrusion, allowing for a wider variety of extruders with lower maximum torque to be used. The other polymers presented herein show the same, advantageous properties as indicated by their similar glass transition temperatures.

TABLE 1
Polymer composition and properties

| | Co-monomer composition | | | | | | | | GPC | |
|---|---|---|---|---|---|---|---|---|---|---|
| | EHMA | EMA | HEMA | TMAEMC | MMA | EA | T_{g} | MFFT | M_{w} | |
| Compound | [%] | [%] | [%] | [%] | [%] | [%] | [° C.] | [° C.] | [Da] | D |
| Polymer 1 | 67.4 | 22.6 | 5 | 5 | | | 23 | 6.5 | 93 300 | 2.04 |
| Polymer 2 | 59.9 | 20.1 | 15 | 5 | | | 22 | 9.5 | 93 700 | 1.90 |
| Polymer 3 | 72.9 | | 5 | 5 | 17.1 | | 24 | 5.5 | 95 200 | 1.92 |
| Polymer 4 | 65.7 | | 15 | 5 | 14.3 | | 23 | 4.5 | 91 300 | 1.94 |
| Polymer 5 | 66.8 | 22.4 | | 10.8 | | | 12 | 21 | 92 900 | 1.82 |
| Polymer 6 | 66.5 | 22.3 | 10 | 1.2 | | | 11 | 25 | 97 200 | 2.06 |
| EUDRAGIT ® RS | | | | 5 | 65 | 30 | 65 | 45.0 | 33 200 | 2.6 |

TABLE 2
Release profiles of matrix tablets prepared by spray granulation and subsequent compression

| | | | | | Polymers 1 + 2 | | | | | | Polymers 6 + 5 | |
|---|---|---|---|---|---|---|---|---|---|---|---|---|
| Polymer | Polymer 1 | | Polymer 2 | | (1:1) | | Polymer 6 | | Polymer 5 | | (1:1) | |
| API | Theophylline | | Theophylline | | Theophylline | | Theophylline | | Theophylline | | Theophylline | |
| | powder | | powder | | powder | | powder | | powder | | powder | |
| Polymer weight gain [% (w/w) | 20 | | 20 | | 20 | | 20 | | 20 | | 20 | |
| compared to drug substrate | | | | | | | | | | | | |
| mass] | | | | | | | | | | | | |
| Active release without/with | | | | | | | | | | | | |
| 40% EtOH (w/w) | | | | | | | | | | | | |
| 5 min (pH 1.2 without/with | 3.19 | 3.91 | 3.88 | 5.55 | 4.56 | 5.27 | 3.81 | 5.1 | 3.70 | 4.5 | 3.92 | 5.27 |
| EtOH) | | | | | | | | | | | | |
| 30 min (pH 1.2 without/with | 10.02 | 13.67 | 11.21 | 13.65 | 11.99 | 13.91 | 10.84 | 23.63 | 10.07 | 12.07 | 10.33 | 15.07 |
| EtOH) | | | | | | | | | | | | |
| 1 h (pH 1.2 without/with | 15.20 | 22.04 | 16.52 | 19.72 | 16.52 | 20.34 | 16.52 | 40.59 | 14.80 | 17.66 | 15.62 | 23.86 |
| EtOH) | | | | | | | | | | | | |
| 1.5 h (pH 1.2 without/with | 19.16 | 28.38 | 20.51 | 24.48 | 20.12 | 25.44 | 20.84 | 49.23 | 18.37 | 22.44 | 19.98 | 32.06 |
| EtOH) | | | | | | | | | | | | |
| 2 h (pH 1.2 without/with | 22.53 | 33.85 | 23.91 | 28.64 | 23.23 | 30.00 | 24.44 | 55.06 | 21.48 | 27.01 | 23.54 | 38.73 |
| EtOH) | | | | | | | | | | | | |
| 2.5 h (pH 6.8 without EtOH) | 25.25 | 37.68 | 26.60 | 31.42 | 25.65 | 33.35 | 27.07 | 58.71 | 23.65 | 30.5 | 26.06 | 42.75 |
| 3 h (pH 6.8 without EtOH) | 27.51 | 40.74 | 28.82 | 33.74 | 27.63 | 36.11 | 29.4 | 61.51 | 25.84 | 33.41 | 28.28 | 46.06 |
| 3.5 h (pH 6.8 without EtOH) | 29.61 | 43.57 | 30.94 | 35.88 | 29.51 | 38.77 | 31.56 | 64.01 | 27.89 | 36.23 | 30.31 | 48.93 |
| 4 h (pH 6.8 without EtOH) | 31.57 | 46.17 | 32.96 | 37.87 | 31.22 | 41.26 | 33.56 | 66.25 | 29.73 | 38.95 | 32.23 | 51.45 |
| 4.5 h (pH 6.8 without EtOH) | 33.41 | 48.58 | 34.80 | 39.72 | 32.81 | 43.61 | 35.41 | 68.35 | 31.47 | 41.49 | 34.00 | 53.81 |
| 5 h (pH 6.8 without EtOH) | 35.16 | 50.81 | 36.60 | 41.43 | 34.33 | 45.84 | 37.16 | 70.28 | 33.07 | 43.93 | 35.65 | 55.99 |
| 5.5 h (pH 6.8 without EtOH) | 36.82 | 52.89 | 38.29 | 43.05 | 35.77 | 47.98 | 38.8 | 72.06 | 34.61 | 46.23 | 37.21 | 57.99 |
| 6 h (pH 6.8 without EtOH) | 38.40 | 54.84 | 39.89 | 44.52 | 37.11 | 50.07 | 40.35 | 73.69 | 36.06 | 48.41 | 38.68 | 59.83 |
| 6.5 h (pH 6.8 without EtOH) | 39.88 | 56.67 | 41.41 | 45.94 | 38.42 | 52.02 | 41.84 | 75.26 | 37.45 | 50.49 | 40.12 | 61.6 |
| 7 h (pH 6.8 without EtOH) | 41.31 | 58.39 | 42.89 | 47.24 | 39.65 | 53.88 | 43.25 | 76.74 | 38.78 | 52.45 | 41.46 | 63.26 |
| 8 h (pH 6.8 without EtOH) | 44.15 | 61.59 | 45.68 | 49.64 | 42.01 | 57.41 | 46.02 | 79.48 | 41.29 | 56.14 | 44.11 | 66.26 |
| 9 h (pH 6.8 without EtOH) | 46.77 | 64.39 | 48.29 | 51.81 | 44.19 | 60.47 | 48.51 | 81.84 | 43.68 | 59.44 | 46.55 | 68.83 |
| 10 h (pH 6.8 without EtOH) | 49.23 | 66.94 | 50.73 | 53.73 | 46.21 | 63.18 | 50.86 | 83.93 | 45.88 | 62.42 | 48.77 | 71.14 |

| | | | | | | |
|---|---|---|---|---|---|---|
| Arithmetic average (bold | 13.84 | 4.26 | 10.43 | 30.83 | 9.71 | 17.92 |
| figures) | | | | | | |
| n for calculation | 17 | 17 | 17 | 17 | 17 | 17 |
| Ethanol resistance | yes | yes | yes | no | yes | yes |

TABLE 3
Release profiles of matrix tablets prepared by spray granulation and subsequent compression if not otherwise mentioned

| Polymer | EUDRAGIT ® RS | | Polymer 3 | | Polymer 4 | | Polymer 2 | |
|---|---|---|---|---|---|---|---|---|
| API | Theophylline | | Theophylline | | Theophylline | | Theophylline powder | |
| | powder | | powder | | powder | | | |
| Comment | | | | | | | Prepared by direct compression (see example 5) | |
| Polymer weight gain [% (w/w) | 20 | | 20 | | 20 | | 20 | |
| compared to drug substrate mass] | | | | | | | | |
| Active release without/with 40% | | | | | | | | |
| EtOH (w/w) | | | | | | | | |
| 5 min (pH 1.2 without/with EtOH) | 4.16 | 11.71 | 8.19 | 8.30 | 3.05 | 3.59 | 3.73 | 4.50 |
| 30 min (pH 1.2 without/with EtOH) | 18.9 | 61.94 | 24.56 | 28.50 | 9.05 | 10.66 | 10.88 | 12.63 |
| 1 h (pH 1.2 without/with EtOH) | 30.91 | 95.3 | 40.22 | 44.99 | 13.46 | 16.51 | 15.93 | 18.71 |
| 1.5 h (pH 1.2 without/with EtOH) | 39.81 | 100.04 | 52.92 | 57.45 | 16.89 | 21.32 | 19.75 | 23.43 |
| 2 h (pH 1.2 without/with EtOH) | 46.44 | 99.69 | 63.01 | 67.51 | 19.84 | 25.87 | 22.96 | 27.51 |
| 2.5 h (pH 6.8 without EtOH) | 51.51 | 99.76 | 70.03 | 72.63 | 22.06 | 29.67 | 25.33 | 30.11 |
| 3 h (pH 6.8 without EtOH) | 55.59 | 99.76 | 75.41 | 76.41 | 24.13 | 32.81 | 27.46 | 32.30 |
| 3.5 h (pH 6.8 without EtOH) | 58.92 | 99.76 | 79.62 | 79.25 | 26.08 | 35.65 | 29.41 | 34.29 |
| 4 h (pH 6.8 without EtOH) | 61.75 | 99.78 | 82.99 | 81.50 | 27.91 | 38.21 | 31.21 | 36.12 |
| 4.5 h (pH 6.8 without EtOH) | 64.19 | 99.81 | 85.74 | 83.36 | 29.61 | 40.58 | 32.93 | 37.78 |
| 5 h (pH 6.8 without EtOH) | 66.49 | 99.79 | 88.07 | 85.13 | 31.24 | 42.74 | 34.55 | 39.26 |
| 5.5 h (pH 6.8 without EtOH) | 68.54 | 99.8 | 90.01 | 86.88 | 32.75 | 44.75 | 36.08 | 40.64 |
| 6 h (pH 6.8 without EtOH) | 70.4 | 99.83 | 91.64 | 88.62 | 34.22 | 46.65 | 37.56 | 41.92 |
| 6.5 h (pH 6.8 without EtOH) | 72.07 | 99.84 | 93.10 | 90.26 | 35.6 | 48.44 | 38.97 | 43.11 |
| 7 h (pH 6.8 without EtOH) | 73.58 | 99.85 | 94.45 | 91.86 | 36.93 | 50.14 | 40.33 | 44.23 |
| 8 h (pH 6.8 without EtOH) | 76.3 | 99.9 | 96.97 | 94.99 | 39.45 | 53.31 | 42.88 | 46.30 |
| 9 h (pH 6.8 without EtOH) | 78.61 | 99.91 | 99.00 | 97.59 | 41.81 | 56.19 | 45.48 | 48.18 |
| 10 h (pH 6.8 without EtOH) | 80.64 | 99.95 | 100.12 | 99.19 | 44.01 | 58.80 | 47.68 | 49.89 |

| | | | | |
|---|---|---|---|---|
| Arithmetic average (bold figures) | 38.8 | 10.35 | 3.94 | 3.00 |
| n for calculation | 16 | 16 | 17 | 7 |
| Ethanol resistance | no | yes | yes | Yes |

TABLE 4
Complex viscosity of EUDRAGIT ® RS PO
and Polymer 2 in dependence of temperature

| EUDRAGIT ® | | | |
|---|---|---|---|
| RS PO | Complex | Polymer 2 | Complex |
| Temperature/ | viscosity/ | Temperature/ | viscosity/ |
| ° C. | mPa*s | ° C. | mPa*s |
| 70.06 | 336950000 | 70.06 | 78062000 |
| 71.07 | 355940000 | 71.07 | 76223000 |
| 72.08 | 354710000 | 72.08 | 74094000 |
| 73.08 | 329300000 | 73.09 | 71983000 |
| 74.09 | 328630000 | 74.09 | 69909000 |
| 75.10 | 305910000 | 75.10 | 67698000 |
| 76.11 | 300510000 | 76.11 | 65452000 |
| 77.11 | 275540000 | 77.11 | 63328000 |
| 78.12 | 269230000 | 78.12 | 61094000 |
| 79.13 | 249860000 | 79.13 | 58853000 |
| 80.14 | 234830000 | 80.14 | 56691000 |
| 81.14 | 223320000 | 81.15 | 54515000 |
| 82.15 | 205810000 | 82.15 | 52545000 |
| 83.16 | 192120000 | 83.16 | 50551000 |
| 84.17 | 179030000 | 84.16 | 48606000 |
| 85.17 | 166620000 | 85.17 | 46724000 |
| 86.18 | 154670000 | 86.18 | 44918000 |
| 87.18 | 143530000 | 87.19 | 43192000 |
| 88.20 | 133190000 | 88.19 | 41514000 |
| 89.20 | 124540000 | 89.20 | 39899000 |
| 90.21 | 116090000 | 90.21 | 38348000 |
| 91.21 | 108830000 | 91.22 | 36845000 |
| 92.22 | 102480000 | 92.22 | 35401000 |
| 93.23 | 96532000 | 93.23 | 34006000 |
| 94.24 | 91066000 | 94.24 | 32693000 |
| 95.24 | 85952000 | 95.24 | 31410000 |
| 96.25 | 81233000 | 96.25 | 30186000 |
| 97.26 | 76598000 | 97.26 | 28962000 |
| 98.27 | 72323000 | 98.27 | 27828000 |
| 99.28 | 68414000 | 99.27 | 26705000 |
| 100.28 | 64638000 | 100.28 | 25601000 |
| 101.28 | 61036000 | 101.28 | 24528000 |
| 102.29 | 57697000 | 102.29 | 23479000 |
| 103.30 | 54598000 | 103.30 | 22458000 |
| 104.31 | 51745000 | 104.30 | 21437000 |
| 105.31 | 49209000 | 105.31 | 20451000 |
| 106.32 | 46667000 | 106.32 | 19472000 |
| 107.33 | 44239000 | 107.33 | 18528000 |
| 108.33 | 41921000 | 108.33 | 17591000 |
| 109.34 | 39851000 | 109.34 | 16663000 |
| 110.35 | 37983000 | 110.35 | 15786000 |
| 111.35 | 36281000 | 111.35 | 14905000 |
| 112.36 | 34765000 | 112.36 | 14049000 |
| 113.37 | 33347000 | 113.37 | 13225000 |
| 114.38 | 32017000 | 114.38 | 12429000 |
| 115.38 | 30793000 | 115.38 | 11664000 |
| 116.39 | 29629000 | 116.39 | 10929000 |
| 117.39 | 28563000 | 117.39 | 10226000 |
| 118.40 | 27547000 | 118.40 | 9539000 |
| 119.41 | 26731000 | 119.41 | 8898400 |
| 120.41 | 25958000 | 120.42 | 8301100 |
| 121.41 | 25225000 | 121.42 | 7739000 |
| 122.42 | 24534000 | 122.43 | 7207700 |
| 123.43 | 23860000 | 123.43 | 6707000 |
| 124.44 | 23203000 | 124.44 | 6231000 |
| 125.45 | 22580000 | 125.45 | 5778600 |
| 126.45 | 21953000 | 126.46 | 5355600 |
| 127.45 | 21319000 | 127.46 | 4963200 |
| 128.46 | 20613000 | 128.47 | 4592000 |
| 129.48 | 19900000 | 129.48 | 4251200 |
| 130.49 | 19069000 | 130.49 | 3931000 |
| 131.49 | 18018000 | 131.49 | 3637200 |
| 132.50 | 16963000 | 132.50 | 3360600 |
| 133.51 | 15983000 | 133.51 | 3105600 |
| 134.51 | 15048000 | 134.51 | 2868800 |
| 135.52 | 14190000 | 135.52 | 2651400 |
| 136.52 | 13355000 | 136.52 | 2446000 |
| 137.53 | 12552000 | 137.53 | 2259400 |
| 138.54 | 11801000 | 138.53 | 2086000 |
| 139.55 | 11081000 | 139.54 | 1927900 |
| 140.55 | 10421000 | 140.55 | 1781400 |
| 141.56 | 9766300 | 141.56 | 1646500 |
| 142.56 | 9161800 | 142.56 | 1522100 |
| 143.57 | 8585500 | 143.57 | 1408500 |
| 144.58 | 8041000 | 144.58 | 1304600 |
| 145.59 | 7517800 | 145.59 | 1208900 |
| 146.59 | 7025100 | 146.59 | 1121800 |
| 147.60 | 6552700 | 147.60 | 1041700 |
| 148.61 | 6102600 | 148.60 | 967200 |
| 149.62 | 5677900 | 149.61 | 899020 |
| 150.62 | 5267400 | 150.62 | 836680 |
| 151.62 | 4877600 | 151.63 | 779930 |
| 152.63 | 4512100 | 152.62 | 726800 |
| 153.64 | 4167900 | 153.64 | 678740 |
| 154.64 | 3841900 | 154.65 | 633860 |
| 155.65 | 3525200 | 155.66 | 593360 |
| 156.66 | 3226900 | 156.66 | 556440 |
| 157.67 | 2944500 | 157.66 | 520820 |
| 158.67 | 2678700 | 158.67 | 488600 |
| 159.68 | 2425800 | 159.68 | 458100 |
| 160.68 | 2187900 | 160.69 | 430990 |
| 161.69 | 1961900 | 161.69 | 404730 |
| 162.70 | 1754100 | 162.70 | 379490 |
| 163.70 | 1563400 | 163.70 | 356900 |
| 164.70 | 1384900 | 164.71 | 335280 |
| 165.71 | 1219100 | 165.72 | 315210 |
| 166.72 | 1067800 | 166.72 | 295650 |
| 167.74 | 931400 | 167.73 | 277330 |
| 168.74 | 809670 | 168.74 | 259480 |
| 169.75 | 703520 | 169.74 | 242230 |
| 170.75 | 607500 | 170.75 | 225930 |
| 171.76 | 523840 | 171.76 | 209630 |
| 172.76 | 450820 | 172.76 | 194860 |
| 173.77 | 386480 | 173.77 | 179380 |
| 174.78 | 332900 | 174.78 | 165510 |
| 175.78 | 288080 | 175.79 | 150680 |
| 176.79 | 250840 | 176.79 | 137110 |
| 177.79 | 220030 | 177.79 | 123770 |
| 178.80 | 194140 | 178.80 | 110740 |
| 179.80 | 172810 | 179.81 | 99085 |
| 180.81 | 153890 | 180.82 | 88591 |
| 181.82 | 138320 | 181.82 | 79443 |
| 182.83 | 124830 | 182.82 | 71037 |
| 183.83 | 112550 | 183.83 | 64184 |
| 184.84 | 102240 | 184.84 | 58839 |
| 185.84 | 92768 | 185.85 | 54428 |
| 186.85 | 86480 | 186.85 | 50603 |
| 187.86 | 78757 | 187.85 | 46831 |
| 188.86 | 73593 | 188.86 | 44067 |
| 189.87 | 69521 | 189.86 | 41268 |
| 190.88 | 65301 | 190.87 | 39094 |
| 191.88 | 62335 | 191.87 | 36796 |
| 192.87 | 59728 | 192.87 | 35662 |
| 193.87 | 53861 | 193.87 | 33991 |
| 194.88 | 46992 | 194.87 | 32365 |
| 195.88 | 41625 | 195.88 | 30978 |
| 196.89 | 36391 | 196.87 | 29410 |
| 197.89 | 32500 | 197.86 | 28265 |
| 198.88 | 29340 | 198.85 | 27490 |
| 199.87 | 26680 | 199.84 | 26627 |
| 200.75 | 24396 | 200.75 | 26064 |

Comparing the complex viscosity of EUDRAGIT® RS P0 (comparative, not according to the invention) to the complex viscosity of freeze-dried polymer 2 (according to the invention) shows that EUDRAGIT® RS P0 requires temperatures that are about 20° C. higher to reach a comparable complex viscosity as Polymer 2. For example, for Polymer 2, complex viscosities below 10⁷ mPa*s are already reached at above 118° C. while this requires temperatures of 140° C. or more for EUDRAGIT® RS PO.

1: A dosage form, comprising a core, comprising a polymeric matrix, comprising one or more polymer(s) and a biologically active ingredient,
wherein the polymeric matrix comprises 10% by weight or more of the one or more polymer(s) and wherein the one or more polymer(s) are polymerized from a monomer mixture comprising the monomers:
(a) 70 to 95% by weight of 2-ethylhexyl methacrylate (EHMA) and ethyl methacrylate (EMA), or 2-ethylhexyl methacrylate (EHMA) and methyl methacrylate (MMA),
(b) 0 to 25% by weight of a C₂ to C₆ hydroxy-alkylester of acrylic acid or methacrylic acid, and
(c) 2.5 to 20% by weight of a C₂ to C₈ alkyl ester of acrylic acid or of methacrylic acid with a quaternary cationic group in the alkyl group.

2: The dosage form according to claim 1, wherein the monomers (a) comprise 2-ethylhexyl methacrylate (EHMA) and ethyl methacrylate (EMA), or 2-ethylhexyl methacrylate (EHMA) and methyl methacrylate (MMA), at a ratio by weight from 5:1 to 1:1.

3: The dosage form according to claim 1, wherein the monomers (b) are selected from the group consisting of 2 hydroxyethyl methacrylate (HEMA), 2-hydroxypropyl methacrylate, 3-hydroxypropyl methacrylate, 2,3-dihydroxy propyl methacrylate, 2 hydroxyethyl acrylate, 2-hydroxypropyl acrylate, 3-hydroxy propyl acrylate, 2,3-dihydroxypropyl acrylate, and any mixtures thereof.

4: The dosage form according to claim 1, wherein the monomers (c) comprises 2-trimethylammonium ethyl methacrylate Chloride (TMAEMC), 2-trimethylammonium propyl methacrylate chloride (TMAPMC), or both.

5: The dosage form according to claim 1, wherein the one or more polymer(s) are polymerized from a monomer mixture comprising (a1), (a2), (b) and (c);
(a1) 40 to 80% by weight of 2-ethylhexyl methacrylate,
(a2) 10 to 30% by weight of 2-ethyl methacrylate or methyl methacrylate or both,
(b) 0 to 20% by weight of 2 hydroxyethyl methacrylate, and
(c) 2.5 to 20% by weight of 2-trimethylammonium ethyl methacrylate chloride, wherein (a1), (a2), (b), and (c) add up to 90 to 100%.

6: The dosage form according to claim 1, wherein the one or more polymer(s) have a minimum film forming temperature (MFFT) of 35° C. or lower.

7: The dosage form according to claim 1, wherein the one or more polymer(s) have a midpoint glass transition temperature (T_{mg}) in a range from 0 to 50° C.

8: The dosage form according to claim 1, wherein the one or more polymer(s) have a weight average molecular weight Mw from 10,000 to 200,000 Dalton.

9: The dosage form according to claim 1, wherein the polymeric matrix comprises 10 to 99% by weight of the one or more polymer(s), 1 to 90% by weight of the biologically active ingredient, and 0 to 89% by weight of pharmaceutically or nutraceutically acceptable excipients.

10: The dosage form according to claim 9, wherein the pharmaceutically or nutraceutically acceptable excipients are selected from the group consisting of antioxidants, brighteners, binding agents, flavoring agents, flow aids, glidants, penetration-promoting agents, pigments, plasticizers, further polymers, pore-forming agents, stabilizers and any combinations thereof.

11: The dosage form according to claim 1, which is an orally delivered medicament with resistance against an influence of ethanol.

12: A process for preparing the dosage form according to claim 1, the process comprising:
mixing the one or more polymer(s), the biologically active ingredient and optionally pharmaceutically acceptable excipients, and
processing the mixture by dry granulation, powder compression, spray granulation, wet granulation and extrusion or melt extrusion, or comminution to granulates or powders; to a final dosage form in the form of pellets, beads, tablets, sachets or capsules filled with pellets, beads, granulates or powders.

13. (canceled)

14: The dosage form according to claim 4, wherein the monomers (c) comprise 2-trimethylammonium ethyl methacrylate chloride (TMAEMC).
